(19) United States
(12) Patent Application Publication
(10) Pub. No.: US 2025/0121167 A1
(43) Pub. Date: Apr. 17, 2025

(54) Microneedle Device and Method for Detecting At Least One Force Acting on a Microneedle Array

(71) Applicant: LTS Lohmann Therapie-Systems AG, Andernach (DE)

(72) Inventor: Maral Heidary Dastjerdi, Remagen (DE)

(21) Appl. No.: 18/683,548

(22) PCT Filed: Jul. 25, 2022
(86) PCT No.: PCT/EP2022/070788

§ 371 Date: Feb. 14, 2024

(30) Foreign Application Priority Data
Aug. 19, 2021 (DE) 10 2021 121 528.5

(51) Int. Cl. A61M 37/00

(52) U.S. Cl. CPC A61M 37/0015; A61M 2037/0061; A61M 2205/332

(57) ABSTRACT

The invention relates to a microneedle device, in particular a manually applied microneedle device. The microneedle device has a microneedle array and a sensor device connected to the microneedle array. The sensor device is designed for detecting at least one force acting on the microneedle array. The invention also relates to a method for detecting at least one force acting on a microneedle array by means of a sensor device connected to the microneedle array. The invention further relates to a method for applying a microneedle array.

### CROSS-REFERENCE TO RELATED APPLICATIONS

This application is the United States national phase of International Patent Application No. PCT/EP2022/070788 filed Jul. 25, 2022, and claims priority to German Patent Application No. 10 2021 121 528.5 filed Aug. 19, 2021, the disclosures of which are hereby incorporated by reference in their entireties.

### BACKGROUND OF THE INVENTION

### Field of the Invention

The invention relates to a microneedle device and method for detecting at least one force acting on a microneedle array.

### Description of Related Art

Beside classic forms of active ingredient administration, such as for example swallowing medicaments, injecting medicine or the like, there are concepts for an application of active ingredients via the skin. In this context, the use of microneedles is of particular interest.

Microneedle arrays, also referred to as microarrays, have a plurality of microneedles that are typically arranged on a support element, such as a patch, a plaster or the like, or connected to a support element. Such microneedle arrays include a high number of microneedles, for example 100 to 600 needles per cm². The needles have a short length, so that upon being pressed into a patient's skin, the needles penetrate the skin only so far that nerves and blood vessels make no contact with needle tips, if possible. The microneedles comprise an active ingredient or a medicament. The corresponding active ingredient may be provided on a surface of the needles or be provided in the needles. It is preferred that the needles are made of a material dissolving in the skin.

For a successful application of a microneedle array, in particular for a successful medication with microneedle arrays, it is decisive that a correct introduction of the microneedle array into the desired skin layer is performed. Here, it is of particular importance that a sufficient insertion depth is achieved upon application.

DE 10 2014 201 605 A1 describes a microneedle device with a container that bursts when a certain contact pressure is applied and releases a colored liquid indicating a successful application. Due to the size and/or the design, it is disadvantageous that this indication of an application is visibly only after use.

Another factor of a successful application of microneedle arrays is the application time. As such, a sufficiently long application has to be made to release the active substance. In particular, the combination of insertion depth and application time is decisive, or, in other words, whether the application has been performed for sufficient time with sufficient insertion depth.

Moreover, skin-specific factors are relevant for the application, in particular for an optimum insertion depth and/or application time. As such, there is a great variance in the specific skin of a respective user, for example due to age, skin texture, illnesses, BMI etc. These factors have an influence, for example, on the force required to reach the insertion depth.

### SUMMARY OF THE INVENTION

It is an object of the invention to provide a microneedle device and a method enabling an improved check of the application of a microneedle array performed.

According to the invention, the object is achieved by a microneedle array as described herein and a method as described herein.

The microneedle array according to the invention is in particular a microneedle array for manual application. Here, manually preferably means that the microneedle array of the microneedle device can be applied manually by a user, in particular using one or more fingers or the hand. The microneedle device is preferably designed for application by means of the thumb of a user. Accordingly, it is preferred that no, in particular no force-applying application device is required for application. On the other hand, the microneedle device may alternatively be a microneedle device to be applied using an in particular force-applying application device. The microneedle device comprises a microneedle array. The microneedle array comprises a plurality of microneedles. The microneedles are preferably in the shape of a cone, a pyramid or an obelisk. The microneedles are connected, in particular integrally, to a support element. The microneedle device further includes a sensor device connected, e.g. adhesively, to the microneedle array. The sensor device is indirectly or directly connected to the microneedle array. It is preferred that the sensor device is connected to the side opposite the tips of the microneedles, which may also be referred to as the rear side. Preferably, the sensor device is connected to the support element, in particular directly. The sensor device is configured to detect at least one force acting on the microneedle array. Preferably, the sensor device is configured to detect at least one force acting on the microneedle array in a direction substantially perpendicular to the microneedle array. As an alternative or in addition to the force to be detected, the sensor device is configured to detect at least one pressure acting on the microneedle array. It is preferred that the sensor device is configured to measure the at least one force acting on the microneedle array so that in particular a measuring detection of forces of different magnitudes is performed. The sensor device measures the at least one force in particular as a value with the unit N (Newton) or Pa (Pascal). The sensor device and the microneedle array are preferably connected in a force-fitting and/or a form-fitting and/or a bonded manner. In the case of a bonded connection, it is preferred that the microneedle array and the sensor device are glued and/or soldered and/or welded to each other. The sensor device and the microneedle array are in particular inseparably connected, i.e. they are preferably inseparable by non-destructive methods. Thus. it is preferred that the sensor device and the microneedle array can be separated only by such separation as, for example, sawing, milling and/or breaking. The sensor device and the microneedle array are in particular permanently connected. The sensor device and the microneedle array are preferably rigidly connected to one another.

It is preferred that the sensor device is configured to detect an application force and/or to detect a counterforce. The application force to be detected is in particular a force applied by the user, either indirectly of directly, on the microneedle array, preferably the support element, for the application of the microneedle array into the skin. The counterforce is in particular a force counteracting the application force. Preferably, the counter force is a force acting from the skin on the microneedle array, preferably the microneedles. In particular, the counterforce is dependent on the respective skin selected for the application. Generally, it can be assumed that the counterforce increases during application, until the microneedle array has penetrated the skin. Thereafter, the counterforce decreases until the microneedle array is applied, in particular completely. As soon as no more application force is applied, it can be assumed that the counterforce of the skin pushes the microneedle array out of the skin. Thus, the microneedle device of the present invention advantageously enables skin-specific conditions to be detected by detecting the counterforce, and in particular to adapt the application, preferably the application force, thereto.

It is preferred that the sensor device comprises or is preferably formed by at least one force sensor. It is particularly preferred that the at least one force sensor is a piezo-electric force sensor. In particular, the force sensor is a piezo disc. The piezo disc may be of the type EPZ-20MS64W, for example. The piezo disc is preferably round.

It is preferred that the sensor device comprises two force sensors, in particular two piezo discs. Preferably, one force sensor is configured and/or arranged to detect the application force and one force sensor is configured and/or arranged to detect the counterforce.

It is preferred that the sensor device comprises a support structure. The support structure is in particular of cylindrical design. The cylindrical shape of the support structure can describe a circle, an ellipse, a square, a rectangle or a triangle. It may preferably be a hollow cylinder. The support structure is configured in particular for the transmission of force and/or the accommodation of the at least one force sensor. It is preferred that at least two, preferably all of the following conditions are met: the base of the cylinder shape of the support structure corresponds substantially to the area of the support element; the base of the cylinder shape of the support structure corresponds substantially to the area of the at least one force sensor; the area of the support element corresponds substantially to the area of the at least one force sensor. Preferably, a force sensor is connected, in particular directly, to a base of the support structure. Preferably, in particular the other base of the support structure is connected, either directly or indirectly, to the support element of the microneedle array. The support structure and/or the sensor device are in particular substantially of rotationally symmetrical design.

It is preferred that two force sensors are connected to the support structure opposite each other. Here, the two force sensors are in particular spaced apart by the support structure. Preferably, one of the force sensors is in particular directly connected to a base of the support structure, while the other force sensor is in particular directly connected to the other base of the support structure. One of the force sensors is preferably connected to the support structure by one side and to the microneedle array, in particular the carrier element, by the other side.

It is preferred that the sensor device is connected to the rear side of the microneedle array, in particular in a two-dimensional manner. In particular, the rear side is the surface of the support element. Preferably, a force sensor or the support structure is connected to the support element.

It is preferred that the microneedle device includes a processing device for evaluating the at least one detected force. In particular, the processing device comprises a processor. Preferably, the processing device is connected to the sensor device, in particular in an information-transmitting manner. The connection between the processing device and the sensor device is preferably wired and/or wireless, e.g. via Bluetooth or Wifi. It is possible that the processing device is part of a mobile device, such as a smartphone.

It is preferred that the processing device is set up to compare the at least one detected force with a target force. The target force is in particular 10 N to 100 N. In particular, the target force is a, preferably predefined, force that is required for application. Preferably, the target force is a minimum force that is at least required for application. Aa an alternative or in addition, it is preferred that the processing device is configured to record the time the force is applied to the microneedle device, in particular the microneedle array. The processing device is preferably configured to compare the time the force is applied with a target time, in particular a predefined target time for the application. In particular, the target time is 3 s to 300 s. It is particularly preferred that the processing device is configured to record the application force over time and preferably to compare it with a target value. In particular, the processing device is configured to detect when the target force is reached by the detected force and/or when the target time is reached by the application time, where it is preferred that reaching the target force is detected over the period of the target time. The target force has been reached over the target time period if the detected force corresponds to at least the target force over a period of time up to the target time, in particular up to at least the target time. It is preferred that the target force is reached over the period of the target time if the detected force corresponds to the target force in a single time interval, i.e. continuously, or in total, i.e. over a cumulative period, until at least the target time is reached. It is preferred that the processing device is configured to record the detected forces and/or times. It is preferred that the processing device is configured to compare the application force with the counterforce. This makes it possible, for example, to incorporate skin-specific characteristics. Preferably, the processing device is configured to determine the target force for the application force as a function of the counterforce.

It is preferred that the processing device is configured to transmit feedback to a person about an application of the microneedle array carried out using the microneedle device. In particular, the roll is a press roll. Preferably, the processing device detected up to transmit feedback when the target force is reached by the detected force and/or when the target time is reached by the application time. It is particularly preferred that the processing device is configured to transmit a feedback upon reaching the target force over the target time. It is preferred that a single signal, in particular an acoustic and/or optical and/or haptic signal is output upon reaching the target time and/or the application time and/or the target force over the period of the target time.

It is preferred that the microneedle device comprises a feedback device, in particular an acoustic and/or optical and/or haptic feedback device for transmitting, preferably outputting the feedback. The feedback device can, for example, comprise a light device, such as a LED, and/or a speaker. The feedback device can, for example, also comprise an actuator, such as a vibration element. It is preferred that the feedback device is configured to output a feedback upon a successful and/or an unsuccessful application. In particular, the feedback output upon a successful application differs from the one output upon an unsuccessful application. Preferably, the feedback device is connected to the processing device', in particular in an information-transmitting manner. The connection between the processing device and the feedback device is preferably wired and/or wireless, e.g. via Bluetooth or Wifi. It is possible that the feedback device is part of a mobile device, such as a smartphone.

It is preferred that the microneedle device comprises a finger application surface for manual application of the microneedle array with at least one finger of a user. The finger application surface preferably is a thumb application surface for application of the microneedle array by means of a thumb. In particular, the finger application surface has substantially the size of a human finger, preferably a thumb. It is preferred that the finger application surface is connected, in particular integrally, with an exposed surface of a force sensor of the microneedle device, preferably corresponds to the same. The finger application surface is preferably planar.

It is preferred that the microneedle device, in particular the sensor device, is configured such that the application force, e.g. the application force applied on the finger application surface, is homogenously transmitted to the microneedle array.

Furthermore, the disclosure also relates to a method for detecting at least one force acting on a microneedle array by means of a sensor device connected to the microneedle array. The detected force is in particular an application force, preferably a manual application force of a user, and/or a counterforce on the microneedle array.

It is preferred that the method for detecting at least one force acting on a microneedle array is performed using a microneedle device having one or a plurality of the features of the above described microneedle device.

The disclosure further relates to a method for applying a microneedle array. The method comprises the step of applying the microneedle array into the skin of a patient. The application is in particular performed manually or by means of an application device. Further, the method comprises the step of detecting, in particular measuring, at least one force acting on the microneedle array by means of the above described method for detecting at least one force acting on a microneedle array. The detected force is in particular an application force, preferably a manual application force of a user, and/or a counterforce on the microneedle array. Optionally, the method comprises the further step of evaluating the at least one detected force. The evaluation comprises in particular a comparison of the detected force with a preferably predefined target force for the application. Another optional step of the method is a feedback output regarding the at least one detected force. It is preferred that the feedback output occurs as soon as the detected force has reached a target force, in particular over a period until a target force is reached. The feedback output is preferably haptic and/or optical and/or acoustic.

It is preferred that the method for applying a microneedle array is performed using a microneedle device having one or a plurality of the features of the above described microneedle device.

In the following, the invention is described in more detail by means of preferred embodiments with reference to the accompanying drawings.

### BRIEF DESCRIPTION OF THE DRAWINGS

The terms Fig., Figs., Figure, and Figures are used interchangeably in the specification to refer to the corresponding figures in the drawings.

In the Figures:

FIG. 1 is a schematic sectional side view of an embodiment of a microneedle device according to the invention during application,

FIG. 2 is a schematic sectional side view of another embodiment of a microneedle device according to the invention,

FIG. 3 is a schematic sectional side view of another embodiment of a microneedle device according to the invention, and

FIG. 4 is a schematic sectional side view of another embodiment of a microneedle device according to the invention.

### DESCRIPTION OF THE INVENTION

In the Figures, similar or identical components or elements are identified by the same reference numerals or variations thereof (e.g. 16 and 16′, 16″). In particular for improved clarity, preferably already identified elements are not provided with reference numerals in all Figures.

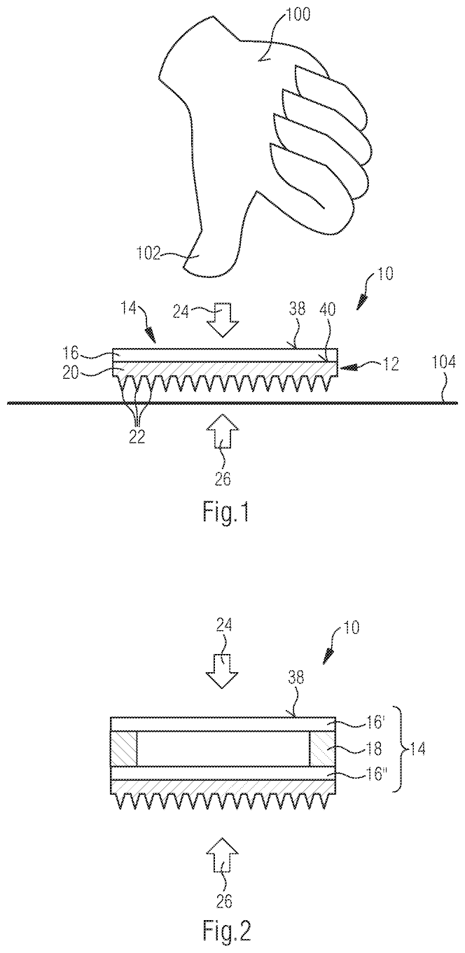

FIG. 1 is a schematic sectional side view of a microneedle device 10 during application.

The microneedle device 10 comprises a microneedle array 12 which has a support element 20, such as a patch, with a plurality of microneedles 22 connected to it, in particular integrally.

A sensor device 14 is connected, e.g. adhesively, to the rear side 40 of the microneedle array 12, which corresponds in this case to the surface of the support element 20. The sensor device 14 is shown as comprising, in particular consisting of a single force sensor 16. In particular, the force sensor is a piezoelectric force sensor 16. It is preferred that the piezoelectric force sensor 16 is a piezo disc, in particular a round disc.

The microneedle device 10 is shown being applied by the thumb 102 of the hand 100 of a user, such as a patient or medical personnel, into the skin 104, for example of a patient. Here, an application force (shown by arrow 24) is applied to an application surface 38 of the microneedle device 10 by means of the thumb 102, thus inserting the microneedles 22 into the skin 104. The application force 24 can, for example, be applied over a period of time so that the microneedle array 12 is pressed further.

The application force 24 can be detected, in particular measured, by the force sensor 16.

The skin 104 acts against the insertion with a counterforce (shown by arrow 26) on the microneedle array 12.

The microneedle array 12 and/or the sensor device 14 have in particular a circular, oval, rectangular or square base. Preferably, the microneedle array 12 and/or the sensor device 14, particularly preferably the microneedle device 10, is symmetrical, in particular rotationally symmetrical.

FIG. 2 shows another embodiment of a microneedle device 10 according to the present invention.

The sensor device 14 is shown as including two, in particular piezoelectric, force sensors 16′, 16″, which are spaced apart by a hollow cylindrical support structure 18. In particular, the support structure 18 has a circular base.

The counterforce 26 can be detected via the force sensor 16″ directly connected to the microneedle array 12, while the application force 24 can be detected via the force sensor 16′ connected, in particular integrally, to the application surface 38.

During an application, the application force 24, e.g. from the thumb 102, acts on the microneedle array 12. This application force 24 increases in particular up to a target force at which the microneedle array 12 penetrates the skin 104. This force, i.e. in particular the application force 24≥target force, is then maintained over a period of time (holding time), for example by the thumb 102. The skin 104 continues to exert the counterforce 26 against the microneedle array 12 and thus, for example, also against the thumb 102. This counterforce 26 increases until the microneedle array 12 penetrates the skin 104. The counterforce 26 then decreases while the microneedle array 12 penetrates further. When the application is ended or canceled, for example when the thumb 102 is removed, in particular the counterforce 26 eventually prevails. In particular, the counterforce 26 is in particular dependent on the respective skin 104 selected for the application. The sensor device 14 with the two force sensors 16′, 16″ can preferably detect the application force 24 and the counterforce 26. This enables an optimum evaluation and/or adaptation of the application.

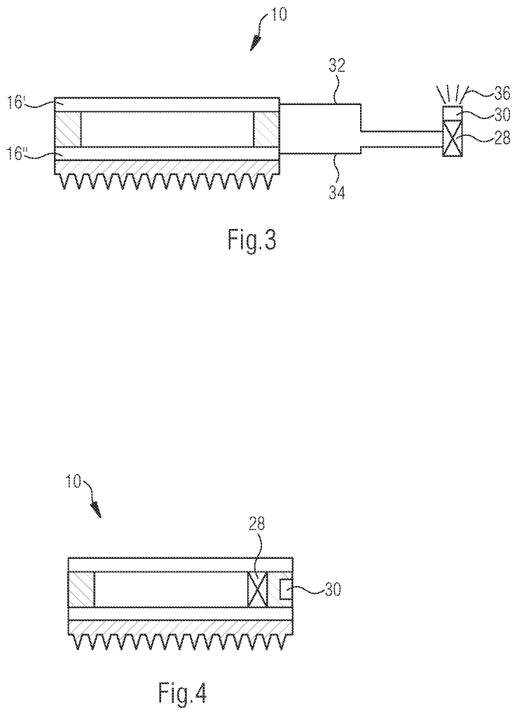

FIG. 3 shows another embodiment of a microneedle device 10, the embodiment being based substantially on the embodiment of FIG. 2.

In FIG. 3, the force sensor 16′ is connected via the cable 32 and the force sensor 16″ is connected via the cable 34 to a processing device 28, in particular in an information-transmitting manner.

The processing device 28 is configured in particular to evaluate the detected values of the force sensors 16′, 16″.

A feedback (represented by dashes 36) can be provided to a user via a feedback device 30 connected to the processing device 28. For example, a feedback can be provided as soon as a sufficient application force 24 is applied, in particular as soon as a sufficient application force 24 has been applied over a preferably predefined period of time.

The feedback device 30 may, for example, have a lamp, such as an LED, and/or a loudspeaker and/or an actuator, such as a vibrating element.

Instead of the wired connection of the sensor device 14 to the processing device 28, a wireless connection, such as a Wifi and/or Bluetooth connection, is also possible, for example.

FIG. 4 shows another embodiment of a microneedle device 10 according to the invention, the embodiment being based substantially on the embodiment of FIGS. 2 and 3.

In contrast to the embodiment shown in FIG. 3 comprising an external processing device 28 and feedback device 30, the processing device 28 and the feedback device 30 are integrated into the sensor device 14 in the embodiment shown in FIG. 4.

1. A microneedle device, in particular a manually applied microneedle device, comprising:
a microneedle array, and
a sensor device connected to the microneedle array,
the sensor device being configured to detect, in particular to measure, at least one force acting on the microneedle array.

2. The microneedle device according to claim 1, wherein the sensor device is configured to detect an application force and/or to detect a counterforce.

3. The microneedle device according to claim 1, wherein the sensor device comprises at least one force sensor, in particular at least one piezoelectric force sensor.

4. The microneedle device according to claim 3, wherein the sensor device comprises two force sensors.

5. The microneedle device according to claim 1, wherein the sensor device comprises an in particular cylindrical support structure.

6. The microneedle device according to claim 5, wherein two opposing force sensors are connected to the support structure.

7. The microneedle device according to claim 1, wherein the sensor device tis connected in particular two-dimensionally to the rear side of the microneedle array.

8. The microneedle device according to claim 1, characterized by a processing device for the evaluation of the at least one detected force.

9. The microneedle device according to claim 8, wherein the processing device is configured to compare the at least one detected force with a target force and/or to detect the period of force application, in particular detecting the force progression over time.

10. The microneedle device according to claim 9, wherein the processing device is configured to provide a person with a feedback on an application of the microneedle array performed using the microneedle device.

11. The microneedle device according to claim 1, characterized by an in particular acoustic and/or optical and/or haptic feedback device.

12. The microneedle device according to claim 1, characterized by a finger application surface for the manual application of the microneedle array by at least one finger of a user.

13. A method for detecting at least one force acting on a microneedle array by means of a sensor device connected to the microneedle array.

14. A method for applying a microneedle array, comprising the steps of:
applying the microneedle array, the application being performed in particular manually or by means of an application device;
detecting, in particular measuring, at least one force acting on the microneedle array using the method according to claim 13; and
preferably evaluating the at least one detected force and/or outputting a feedback on the at least one detected force.

15. (canceled)
